(19) United States
(12) Patent Application Publication
(10) Pub. No.: US 2004/0103903 A1
(43) Pub. Date: Jun. 3, 2004

(54) Surgical surface localizing grid

(76) Inventor: Mark H. Falahee, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/689,123

(22) Filed: Oct. 20, 2003

Related U.S. Application Data
(60) Provisional application No. 60/419,629, filed on Oct. 18, 2002.

(51) Int. Cl. A61B 19/00

(52) U.S. Cl. 128/849

(57) ABSTRACT

A system and method particularly useful in performing minimally invasive surgical procedures includes a thin, flexible surgical film including a radiopaque grid to assist in x-ray visualization. The grid is also preferably visible to the human eye. The grid may be a Cartesian coordinate grid with distance markings, or may be based on an alternative coordinate system. The film preferably includes an adhesive backing and iodine for cut-through incisions.

### REFERENCE TO RELATED APPLICATION

This application claims priority from U.S. Provisional Patent Application Serial No. 60/419,629, filed Oct. 18, 2002, the entire content of which is incorporated herein by reference.

### FIELD OF THE INVENTION

This invention relates generally to surgical procedures and, more particularly, to a localizing grid particularly useful in minimally invasive surgical procedures.

### BACKGROUND OF THE INVENTION

Minimally invasive surgical techniques are increasingly becoming popular for a variety of surgical procedures. Using these approaches, the surgeon makes relatively small and precise incisions which serve as access points for various surgical instruments, visualization devices, and so forth. Compared to standard or “open” cases, minimally invasive procedures enable patients to leave the hospital sooner and, on average, experience less pain, and require less medication during and after hospitalization.

According to current practice, marks are made directly on the skin of a patient under x-ray. After the marks are made, a sterile surgical drape is typically placed over the markings and the incisions are made therethrough to perform the operation. This procedure is considered to be somewhat crude, and frequently results in less than the desired accuracy. Additionally, the initial radiation exposure required to make the markings directly on the skin increases patient exposure, which is undesirable.

### SUMMARY OF THE INVENTION

This invention resides in a system and method particularly useful in performing minimally invasive surgical procedures. In terms of apparatus, the invention includes a thin, flexible surgical film including a radiopaque grid to assist in x-ray visualization. The grid is also preferably visible to the human eye. The grid may be a Cartesian coordinate grid with distance markings, or may be based on an alternative coordinate system. The film preferably includes an adhesive backing and iodine for cut-through incisions.

### BRIEF DESCRIPTION OF THE DRAWING

FIG. 1 is a drawing that shows a preferred embodiment of the invention.

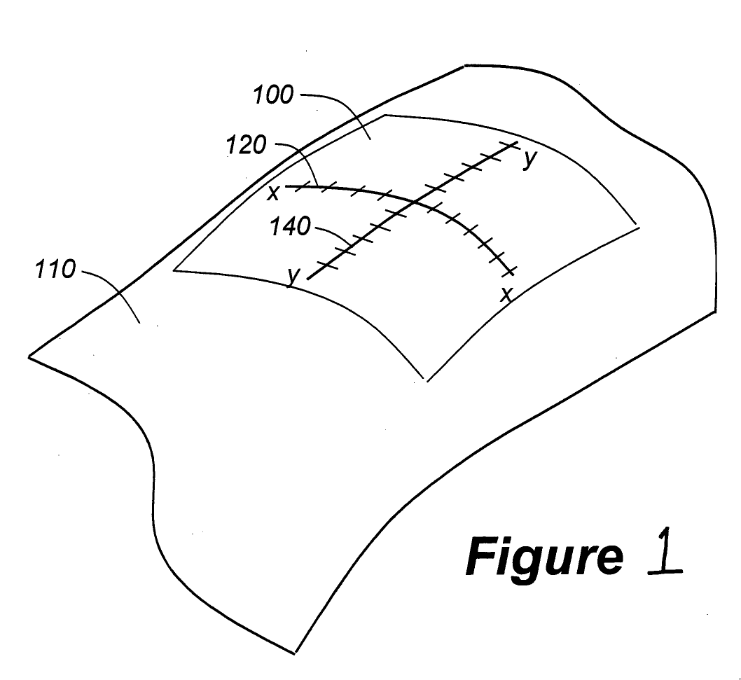

### DETAILED DESCRIPTION OF THE INVENTION

The present invention resides in a sterile covering for use in making precise incisions. The device and method are particularly useful in performing minimally invasive surgical procedures, though not limited to such uses. According to the preferred embodiment, an inventive covering according to the invention includes a sterile film having a patient-contacting surface and an outer surface through which a radiopaque grid is visually apparent for assisting a surgeon in making precise markings and/or incisions at desired instrument access points.

Preferably the grid is visible with to the human eye as well as being radiopaque. The covering is preferably made of a sterile plastic material and includes a Cartesian coordinate radiopaque grid though polar and other coordinate systems may be used. As shown in the FIGURE, the Cartesian coordinate grid may include X and Y axes visible as solid lines, and may further include less prominent increment markings, such as one centimeter.

The covering may include an adhesive backing to provide stability to the covering throughout its use, in which case a release layer would be provided. The covering may be clear, though an iodine-based antiseptic or other material may be used which might cause the film to be translucent as opposed to clear or transparent.

I claim:

1. A sterile covering for use in performing surgical procedures, the covering comprising:
a thin, flexible cut-through film including a radiopaque grid to assist in x-ray visualization.

2. The covering of claim 1, wherein the grid is also visible to the human eye.

3. The covering of claim 1, wherein the grid is a Cartesian coordinate grid.

4. The covering of claim 1, wherein the film includes an adhesive backing.

5. The covering of claim 1, further including distance markings.

6. The covering of claim 1, wherein the film includes iodine or other antiseptic.
